(19) United States
(12) Patent Application Publication
(10) Pub. No.: US 2020/0341010 A1
(43) Pub. Date: Oct. 29, 2020

(54) PREDICTIVE BIOMARKERS FOR TREATMENT OF EYE DISEASES

(71) Applicant: TAMPERE UNIVERSITY FOUNDATION SR, Tampereen yliopisto (FI)

(72) Inventors: Ulla AAPOLA, Tampere (FI); Roger BEUERMAN, Tampere (FI); Antti JYLHÄ, Tampere (FI); Saara LÄHDEKORPI, Tampere (FI); Janika NÄTTINEN, Tampere (FI); Hannu UUSITALO, Tampere (FI)

(21) Appl. No.: 16/956,381

(22) PCT Filed: Dec. 21, 2018
(86) PCT No.: PCT/FI2018/050978

§ 371 Date: Jun. 19, 2020

(30) Foreign Application Priority Data
Dec. 22, 2017 (FI) 20176167

(51) Int. Cl. G01N 33/68

(52) U.S. Cl. CPC G01N 33/6893; G01N 2800/168; G01N 2800/16; G01N 2800/52; G01N 2333/90212; G01N 2333/79; G01N 2333/8139; G01N 2333/948; G01N 2333/4712

(57) ABSTRACT

The present invention relates to a method for predicting a response to a treatment of an eye disease on the basis of proteome changes. The invention also relates to a kit for use the method.

### FIELD OF THE INVENTION

The present invention relates to the field of proteomics. More specifically, the invention relates to means and methods for predicting a response to a treatment of an eye disease on the basis of proteome changes.

### BACKGROUND OF THE INVENTION

The tear film and the underlying ocular surface are the first line of defense in the eye and tears have several purposes in our eyes; they both deliver and take away nutrients and metabolic products from the epithelium of the eye, they lubricate and protect the epithelial surface and participate in delicate control mechanisms. The tear fluid contains many different types of molecules and since the tears reflect the health of the underlying ocular surface and the collection methods are non-invasive, there have been continuous efforts to analyze tears with proteomics in order to identify potential biomarkers for varying diseases. In recent years, proteomics has been utilized in studies of various eye diseases including dry eye, diabetic retinopathy and age-related macular degeneration (AMD).

It is common in proteomics studies to pool samples in order to get more comprehensive answers to clinical questions and this way assume that all patients experience somewhat similar changes in their proteome. This way, individual differences among patients are not often identified. However, drug trials have demonstrated that patients react in different ways to the same treatment and the underlying causes are complex and still largely unknown (Ginsbury & McCarthy, 2001; Schork, 2015). With the latest innovations in the proteomics field, mainly label-free mass spectrometry (MS) methods, it is now possible to treat each patient as an individual in large clinical studies in order to explore the underlying causes for distinct outcome patterns.

Recently, tear proteome has also shown its potential in identifying biomarkers for inflammatory responses associated with glaucoma medication (Funke et al., 2016; Wong et al., 2011). Topical medication is currently the most popular glaucoma management method to lower the intraocular pressure (IOP) and as the condition is incurable, patients are often required to use the eye drops for years or decades. Previous clinical studies have shown that prolonged use of topical glaucoma medication may induce symptoms and signs of ocular surface disease, which overlap to a certain extent with those associated with dry eye disease. The adverse effects could be caused by the active compounds of the eye drops but also by their preservatives such as benzalkonium chloride (BAK)—the most well-known and commonly used preservative in topical glaucoma medication as well as in other topical eye medications. Clinical evidence suggests that patients suffering from adverse reactions whilst using preserved topical treatments benefit from a switch to preservative-free eye drops: their adverse reactions diminish without compromising the control of IOP. The present invention is based on studies on the glaucoma medication switch effects on tear proteome by taking a more personalized approach. Drug trials have demonstrated that patients react in different ways to the same treatment and the underlying causes are complex and largely unknown. There is thus an identified need for means and methods for prediction of an individualized response to a treatment of eye diseases.

### BRIEF DESCRIPTION OF THE INVENTION

An object of the present invention is to provide means and methods of predicting a subject's response to an eye medicament, as well as different uses of the method. This object is achieved by what is stated in the independent claims. Preferred embodiments of the invention are disclosed in the dependent claims.

To be more specific, the present invention provides a method of predicting a response to an eye disease treatment in a subject in need thereof, wherein the method comprises assessing the level of one or more biomarkers comprising PROL1 in a sample obtained from said subject, comparing the assessed level of said biomarkers to a control, and predicting said subject's response to said treatment on the basis of the comparison.

In the method, decreased level of PROL1 is indicative that said subject is likely to benefit from said treatment. In some embodiments, the method comprises assessing a sample obtained from the subject for the level of one or more further biomarkers selected from the group consisting of TF, CST4, HSPA8, CST1, YWHAZ, RNH1, UCHL3, CST2, MYL6, S100A6, B2M, GSR, S100A8, HSPA5, YWHAE, CNDP2, CSTA, CSTB, CST5, and CST3. Preferred further biomarkers are TF and/or MYL6, with or without CTS1.

The invention also provides different uses of the present method. Such uses include selecting a treatment regime to a subject having an eye disease, monitoring response to treatment in a subject having an eye disease, stratifying subjects on the basis of the predicted response to treatment, and stratifying subjects for clinical trials.

Also provided is use of a biomarker combination comprising at least PROL1 and either TF or MYL6, or both TF and MYL6 for determining a subject's response to an eye disease treatment. In some embodiments, the biomarker combination further comprises one or more biomarkers selected from the group consisting of CST4, HSPA8, CST1, YWHAZ, RNH1, UCHL3, CST2, S100A6, B2M, GSR, S100A8, HSPA5, YWHAE, CNDP2, CSTA, CSTB, CST5, and CST3.

The present invention also provides a kit for use in the present method, wherein the kit comprises reagents for specifically assessing the level of at least PROL1 and either TF or MYL6, or both TF and MYL6. In some embodiments, the kit further comprises one or more reagents for specifically assessing the level one or more further biomarkers selected from the group consisting of CST4, HSPA8, CST1, YWHAZ, RNH1, UCHL3, CST2, S100A6, B2M, GSR, S100A8, HSPA5, YWHAE, CNDP2, CSTA, CSTB, CST5, and CST3 in a biological sample, preferably a tear sample. Suitable reagents are readily available in the art and include, for example, antibodies specific for these biomarkers.

Further aspects, specific embodiments, objects, details, and advantages of the invention are set forth in the following drawings, detailed description, and examples.

### BRIEF DESCRIPTION OF THE DRAWINGS

In the following the invention will be described in greater detail by means of preferred embodiments with reference to the attached drawings, in which

FIG. 1 illustrates the study outline summary. During the screening/baseline visit (V1), the patients were switched from preserved latanoprost (L) to preservative-free tafluprost (T) in unit dose dispensers. Clinical measurement together with the tear sample collection were performed at visits V1-V5. At the post-study visit (V6), final clinical measures were recorded but tear samples were no longer collected.

FIG. 2 further expands the explanation of the analysis outline and results from the patient stratification. The top row explains the outline of data processing, protein clustering and patient stratification. The heat map visualizes the change in protein expression between baseline and 12 months after the medication switch. Two protein clusters associated with ocular inflammation are indicated in grey rows, and proteins linked to lacrimal gland secretion in white rows. Two patients were excluded due to missing baseline expression.

FIG. 3 shows proteins (y-axis) that differ at their baseline log₂ expression (x-axis) between the patient groups. All proteins missing an explanation (*, † or #) have a statistical difference between patient groups 1 and 2, and 1 and 3. FIG. 3A shows that protein expression of several pro-inflammatory proteins is highest at baseline for patients in group 1, intermediate in group 2, and lowest in group 3. FIG. 3B shows that protein expression levels of various cystatins, proline-rich protein 1 (PROL1), and beta-2-microglobulin (B2M), considered to be beneficial, are lowest for group 1 patients, intermediate in group 2, and highest in group 3. FIG. 3C displays proteins (all proteins from 3A and 3B included) with differential expressions in comparison to a reference group of elderly patients undergoing strabismus surgery, who do not suffer from glaucoma or DEDlike symptoms associated with topical glaucoma medication.

FIG. 4 shows that Schirmer's test and fluorescein tear break-up time (FTBUT) correlate with statistically significant proteins identified in our study. The black dots represent potential outliers. FIG. 4A shows that TF, S100A6 and YWHAZ protein expression at baseline was high when Schirmer's test results were low. The opposite was true for PROL1, CST4 and B2M. FIG. 4B shows that TF, HSPA8 and YWHAZ protein expression at baseline was high when FTBUT was low.

FIG. 5 illustrates patient examples showing the effects of varying PROL1 (baseline) expression levels. FIG. 5A shows that patients 004 and 028 (squares) had a high baseline expression of PROL1 in comparison to patients 003 and 029 (circles). FIG. 5B shows that the patients with a high baseline level of PROL1, indicated by squares, were not benefiting from the switch based on most clinical signs after 1 year. Lid redness and conjunctival staining increased or remained the same, while corneal staining remained the same. Fluorescein tear break-up time (FTBUT) decreased for patient 004, which also suggests worsening signs. The patients with a low baseline expression of PROL1, indicated by circles, showed signs of decrease inlid redness, corneal staining and conjunctival staining, while FTBUT increased. Conjunctival redness was similar among the patients.

### DETAILED DESCRIPTION OF THE INVENTION

The present invention is based on identification of novel panels or expression profiles of biomarkers that are indicative or predictive of a response to an eye disease therapy in a subject in need of such therapy. Accordingly, an object of the invention is to provide means and methods for various predictive purposes in the treatment of eye diseases.

As used herein, the term “subject” refers to any mammal including, but not limited to, humans and domestic animals such as livestock, pets and sporting animals. Examples of such animals include without limitation carnivores such as cats and dogs and ungulates such as horses. Thus, the present invention may be applied in both human and veterinary medicine. As used herein, the terms “subject”, “patient” and “individual” are interchangeable.

A subject may or may not have been previously diagnosed with an eye disease. Moreover, said subject may be under an eye disease treatment regime or may have previously been under such a regime.

As used herein, the term “eye disease” refers to any disease of the eye where long term topical drug therapy is needed including, but not limited to, any forms of glaucoma, ocular hypertension, ocular surface disease, ocular infections, iritis or uveitis.

As used herein, terms like “eye disease treatment”, “eye disease treatment regime” or “eye disease therapy” refer to any therapy of the eye including but not limited to topical eye drops, ointments, gels or any type of drug delivering implants.

As used herein, the terms “eye medicament” and “eye drug” refer but are not limited to any glaucoma drugs, ocular surface tear substitutes, allergy drugs, corticosteroids or immunomodulators.

As used herein, the term “preserved” eye medicament or drug refers to a formulation which comprises at least one preservative. Non-limiting examples of preservatives typically used in eye formulations include members of quaternary ammonium family like benzalkonium chloride (BAK) and polyquaternium.

As used herein, the term “preservative-free” eye medicament or drug refers to a formulation which does not comprise any preservatives.

As used herein, the term “treatment” or “treating” is intended to include the administration of an eye medicament or drug to a subject for purposes which may include not only complete cure of a disease, but also to alleviation or amelioration of a disease or symptoms related thereto. As used herein, the terms “treatment” and “therapy” are interchangeable.

As used herein, the term “sample” refers to any biological sample, typically a clinical sample, obtainable from an eye of a subject. In some embodiments, a tear sample is the most preferred sample type. Generally, obtaining the sample to be analysed from a subject is not part of the present methods, which may therefore be termed as in vitro methods. Tear samples may be obtained by any appropriate means or methods available in the art, e.g. by collecting tear fluid on an absorbent paper, such as Schirmer's strip or by collecting tears with microcapillary.

The term “sample” also includes samples that have been manipulated or treated in any appropriate way after their procurement, including but not limited to extraction, centrifugation, filtration, precipitation, dialysis, chromatography, treatment with reagents, washing, or enriching for a certain component of the sample.

As used herein, the terms “biomarker” and “marker” are interchangeable and refer to a molecule, preferably a protein, that is differentially present in a sample taken from a subject who will benefit from a given eye disease treatment than in a sample taken from a subject who will not benefit from the same eye disease treatment or who will respond adversely to the same treatment. Thus, the present biomarkers provide information regarding a probable outcome of an eye disease treatment.

Herein, the term “level”, when applied to a biomarker, is used interchangeably with the terms “amount” and “concentration”, and can refer to an absolute or relative quantity of the biomarker in a sample.

In one aspect, the present invention relates to a method of predicting an effect or an outcome of an eye disease treatment in a subject in need thereof. The method comprises providing a sample, preferably a tear sample, obtained from a subject suffering from an eye disease and assessing in said sample the level of at least one of the present biomarkers set forth below and determining said subject's response to a treatment of said eye disease on the basis of said assessment. Preferably, said at least one biomarker comprises PROL1; PROL1 and TF; PROL1 and MYL6; PROL1, TF and MYL6; or PROL1 and CST1.

The method may be used not only for selecting an effective or otherwise beneficial treatment regime (e.g. a certain medication or drug) to a subject, but also to monitor response to treatment. In addition, the method may also be applied for screening new therapeutics for eye diseases. It is envisaged that the present biomarkers may be used for assessing whether or not a candidate drug or intervention therapy is able to modify a biomarker expression profile of a subject suffering from adverse treatment effects towards that of a positive control or towards that of an individual with a favourable treatment response. Furthermore, individuals identified not to respond favourably to a certain treatment on the basis of their biomarker expression profile could be employed as targets in clinical trials aimed for identifying new therapeutic drugs or other intervention therapies for eye diseases. Thus, the present biomarkers may also be used for stratifying individuals for clinical trials.

In some embodiments of the present method of predicting an effect or an outcome of an eye disease treatment in a subject in need thereof, said eye disease treatment involves a drug switch. In such embodiments, the method may be formulated as a method of predicting an effect or an outcome of a drug switch, i.e. a response to drug switch, in the treatment of an eye disease. The method comprises providing a sample, preferably a tear sample, obtained from a subject suffering from said eye disease and assessing in said sample the level of at least one of the present biomarkers set forth below and determining said subject's response to said drug switch on the basis of said assessment. Preferably, said at least one biomarker comprises PROL1; PROL1 and TF; PROL1 and MYL6; PROL1, TF and MYL6; or PROL1 and CST1. In some further embodiments, said drug switch may concern a switch from a preservative-containing, e.g. BAK-containing, eye drug to a preservative-free eye drug.

In some embodiments, the prediction may be based on analysing one or more serial samples obtained from a subject, for example to detect any changes in the response to a particular treatment or combination of treatments for an eye disease. In such instances, the predictive method comprises analysing and comparing at least two samples obtained from the same subject at various time points. The number and interval of the serial samples may vary as desired. The difference between the obtained assessment results serves as an indicator of any change in the response to treatment or as an indicator of effectiveness or ineffectiveness of the treatment or combination of treatments applied.

Determining response to treatment or response to a drug-switch on the basis of a biomarker profile generally requires comparing the assessed or detected levels of biomarkers to a relevant control.

As used herein, the term “control” may refer to a comparable sample obtained from a control subject or a pool of control subjects with a known eye disease history or no history. The term “control” may also refer to a sample previously obtained from the same subject whose response to treatment is to be predicted. In some embodiments, the term “negative control” may refer to a control sample obtained from individuals or pools of individuals who are apparently healthy, and thus, do not show any signs of an eye disease in question or who are known not to benefit form a treatment. In some embodiments, the term “positive control” may refer to a control sample obtained from individuals or pools of individuals who have an eye disease in question or who are known to benefit from a treatment. Accordingly, appropriate control subjects may also include individuals or pools of individuals undergoing or previously treated with a given eye disease treatment. Sometimes it may be beneficial to use more than one type of controls in a single method according to the present invention.

The term “control” may also refer to a predetermined threshold or control value, originating from a single control subject or a pool of control subjects set forth above, which value is indicative of the response to treatment. Statistical methods for determining appropriate threshold or control values will be readily apparent to those of ordinary skill in the art, and the statistically validated threshold or control values can take a variety of forms. For example, a statistically validated threshold can be a single cut-off value, such as a median or mean. Alternatively, a statistically validated threshold can be divided equally (or unequally) into groups, such as low, medium, and high response groups, the low response group being individuals least likely to benefit from the treatment or even likely to have adverse effects, and the high response group being individuals most likely to benefit from the treatment. Furthermore, the threshold may be an absolute value or a relative value. However, if an absolute value is used for the level of the assayed biomarker, then the threshold value is also based upon an absolute value. The same applies to relative values, which must be comparable. In some embodiments, the biomarker levels are normalized using standard methods prior to being compared with a relevant control.

In some embodiments, subjects of the same age, demographic features, and/or disease status, etc. may be employed as appropriate control subjects for obtaining comparable control samples or determining a statistically validated threshold value.

The levels of the assayed biomarkers in a sample obtained from a subject whose response to treatment is to be predicted may be compared with one or more single control values or with one or more ranges of control values, regardless of whether the control value is a predetermined value or a value obtained from a control sample upon practicing the present method. Significance of the difference of biomarker levels in the patient sample and the control can be assessed using standard statistical methods.

Biomarkers for use in the present invention include one or more biomarkers selected from the group consisting of TF, CST4, HSPA8, PROL1, CST1, YWHAZ, RNH1, UCHL3, CST2, MYL6, S100A6, B2M, GSR, S100A8, HSPA5, YWHAE, CNDP2, CSTA, CSTB, CST5, and CST3 in any combination. Depending on the patient group in question, the level of these biomarkers may be either increased, normal or decreased as compared to a relevant control.

As used herein, the term “increased level” refers to an increase in the amount of a biomarker in a sample as compared with a relevant control. Said increase can be determined qualitatively and/or quantitatively according to standard methods known in the art. The term “increased” encompasses an increase at any level, but refers more specifically to an increase between about 1.25 fold and about 10 fold as compared with a relevant control. In some embodiments, the expression is increased if the amount or level of the biomarker in the sample is, for instance, at least about 1.25 times, 1.5 times, 2 times, 3 times, 4 times, 5 times, 6 times, 8 times, 9 times or 10 times, the amount of the same biomarker in the control sample. Said increase can be determined qualitatively and/or quantitatively according to standard methods known in the art. In some embodiments, the term “increased level” refers to a statistically significant increase in the level or amount of the biomarker as compared with that of a relevant control.

As used herein, the term “normal” refers to a detected or assayed biomarker level that is essentially the same or essentially non-altered as compared with that of a relevant control sample or a predetermined threshold value. The term “normal” is interchangeable with the term “non-altered”.

As used herein, the term “decreased level” refers to a decrease in the amount of a biomarker in a sample as compared with a relevant control. Said decrease can be determined qualitatively and/or quantitatively according to standard methods known in the art. The term “decreased” encompasses a decrease at any level, but refers more specifically to a decrease between about 1.25 times and about 10 times as compared with a relevant control. In some embodiments, the expression is decreased if the amount of the biomarker in the sample is, for instance, at least about 1.25 times, 1.5 times, 2 times, 3 times, 4 times, 5 times, 6 times, 8 times, 9 times or 10 times lower than the amount of the same biomarker in the control sample. In some embodiments, the term “decreased level” refers to a statistically significant decrease in the level or amount of the biomarker as compared with that of a relevant control.

As used herein, the expression “indicative of favourable response” and any equivalent expressions include instances where it is likely that the subject will benefit from an eye disease treatment significantly (denoted herein as group 1 patients). In some embodiments, the expression refers to a biomarker which, using routine statistical methods setting confidence levels at a minimum of 95%, is prognostic for favourable response such that the biomarker is found at a certain level (increased, normal or decreased) more often in subjects who will benefit from the treatment than in subjects who will not benefit from the treatment. Preferably, a prognostic biomarker which is indicative of a favourable response is found at a certain level in at least 80% of subjects who will benefit from the treatment, and is found at that level in less than 10% of subjects who will not benefit from the treatment. More preferably, a prognostic biomarker which is indicative of favourable response is found at a certain level in at least 90%, at least 95%, at least 98%, or more in subjects who will benefit from the treatment, and is found at that level in less than 10%, less than 8%, less than 5%, less than 2.5%, or less than 1% of subjects who will not benefit from the treatment.

As used herein, the expression “indicative of non-favourable response” and any equivalent expressions include instances where it is likely that the subject will benefit from an eye disease treatment only to some extent (denoted herein as group 2 patients) or will not benefit from an eye disease treatment or the subject is likely to experience adverse effects caused by the eye disease treatment (denoted herein as group 3 patients). In some embodiments, the expression refers to a biomarker which, using routine statistical methods setting confidence levels at a minimum of 95%, is prognostic for non-favourable response such that the biomarker is found at a certain level (increased, normal or decreased) significantly more often in subjects who will not benefit from the treatment than in subjects who will benefit from the treatment. Preferably, a prognostic biomarker which is indicative of a non-favourable response is found at a certain level in at least 80% of subjects who will not benefit from the treatment, and is found at that level in less than 10% of subjects who will benefit from the treatment. More preferably, a prognostic biomarker which is indicative of non-favourable response is found at a certain level in at least 90%, at least 95%, at least 98%, or more in subjects who will not benefit from the treatment, and is found at that level in less than 10%, less than 8%, less than 5%, less than 2.5%, or less than 1% of subjects who will benefit from the treatment.

Accordingly, in some embodiments of the present invention, subjects may be stratified on the basis of their biomarker expression profiles as those who are likely to have a clear favourable response to a treatment or benefit significantly from said treatment (group 1 patients), or as those who are likely to have a favourable response but to a lesser extent or benefit slightly from said treatment (group 2 patients), or as those who are likely to have a non-favourable response to a treatment or experience adverse effects caused by said treatment (group 3 patients). In other words, by looking into the proteomic profiles it is possible to stratify the patients and predict, which patients would benefit most from the treatment or intervention.

As already set forth above, subjects who are likely to have a clear favourable response to a treatment or benefit significantly from said treatment (i.e. group 1 patients) may in some embodiments be referred to as subjects who are likely to have a favourable response or benefit from said treatment. In such embodiments, subjects who are likely to have a favourable response but to a lesser extent or benefit slightly from said treatment (i.e. group 2 patients) and those who are likely to have a non-favourable response to a treatment or experience adverse effects caused by said treatment (i.e. group 3 patients) may be grouped together and be denoted simply as subjects who are likely not to have a favourable response or not benefit from said treatment. This applies to all further embodiments disclosed herein even if not specifically mentioned in connection with those embodiments.

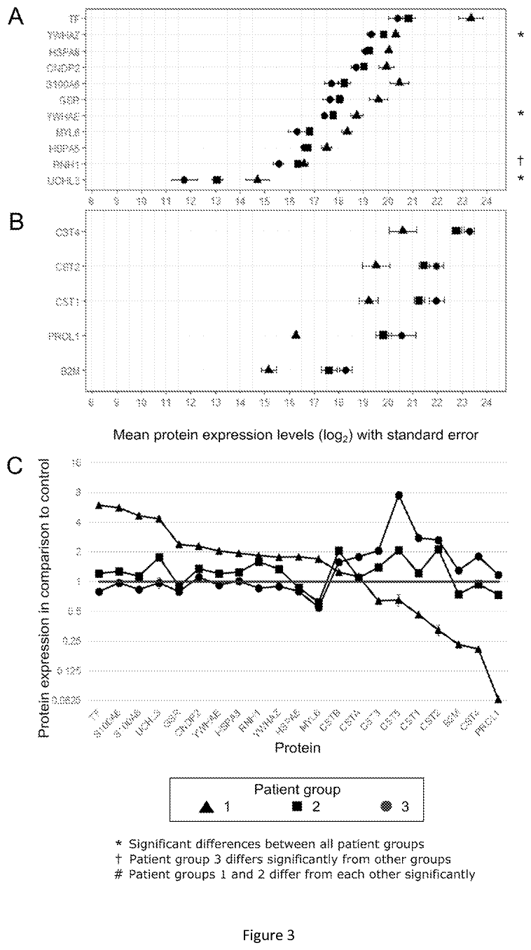

As shown in FIG. 3C, decreased level of PROL1 is indicative of clear favourable response, while normal level of PROL1 indicates that the patient is likely to benefit from the treatment to some extent or that the subject is likely not to benefit from the treatment or even experience adverse effects. Thus, PROL1 may be employed for distinguishing patients of group 1 from the patients of group 2 or 3. Since PROL1 showed the highest fold change between the patient groups among the biomarkers identified herein, it may at least in some embodiments be considered as the best biomarker to be used in the present method.

As shown in FIG. 3C, increased level of TF is indicative of clear favourable response, while normal or decreased level of TF indicates that the patient is likely to benefit from the treatment to some extent or that the subject is likely not to benefit from the treatment or even experience adverse effects. Thus, TF may be employed for distinguishing patients of group 1 from the patients of group 2 or 3. Since TF showed the second highest fold change between the patient groups among the biomarkers identified herein, it may at least in some embodiments be considered as the second best biomarker to be used in the present method.

As shown in FIG. 3C, increased level of MYL6 is indicative of clear favourable response, while normal or decreased level of MYL6 indicates that the patient is likely to benefit from the treatment to some extent or that the subject is likely not to benefit from the treatment or even experience adverse effects. Thus, MYL6 may be employed for distinguishing patients of group 1 from the patients of group 2 or 3. Since MYL6 showed the clearest fold change difference among the biomarkers identified herein, it may at least in some embodiments be considered as the third best biomarker to be used in the present method.

Thus, in some preferred embodiments, the present method is based on determining the level of PROL1 either alone or in combination with TF and/or MYL6. Patients that are likely to have a clear favourable response show decreased expression of PROL1 and increased expression of TF and MYL6. In some further embodiments, the method may comprise assaying a sample obtained from the subject for one or more further biomarkers selected from the group consisting of CST4, HSPA8, CST1, YWHAZ, RNH1, UCHL3, CST2, S100A6, B2M, GSR, S100A8, HSPA5, YWHAE, CNDP2, CSTA, CSTB, CST5, and CST3 in any combination.

As shown in FIG. 3C, decreased level of CST1 is indicative of clear favourable response, while normal level of CST1 indicates that the patient is likely to benefit from the treatment to some extent, while increased level of CST1 indicates that the patient is likely not to benefit from the treatment or to experience adverse effects. Thus, CST1 may be employed for distinguishing patients of groups 1, 2 and 3 from each other.

In some preferred embodiments, the present method is based on determining the level of both PROL1 and CST1. Patients that are likely to have a clear favourable response show decreased expression of both PROL1 and CST1. Patients that are likely to have favourable response but to a lesser extent show normal levels both PROL1 and CST1. Patients that are likely not to benefit from the treatment or to experience adverse effects show normal level of PROL1 and increased level of CST1. In some further embodiments, TF and/or MYL6 are also employed and used in combination with PROL1 and CST1. In some still further embodiments, the method may comprise assaying a sample obtained from the subject for one or more further biomarkers selected from the group consisting of CST4, HSPA8, YWHAZ, RNH1, UCHL3, CST2, S100A6, B2M, GSR, S100A8, HSPA5, YWHAE, CNDP2, CSTA, CSTB, CST5, CST3.

Accordingly, in addition to basing the prediction on PROL1 as set forth above, a subject who is likely to have a significant benefit from the treatment, such as a drug switch (e.g. from a preservative-containing eye drug to preservative-free eye drug) (i.e. patient group 1) may be identified by increased level of one or more biomarkers selected from the group consisting of TF, HSPA8, YWHAZ, RNH1, UCHL3, MYL6, S100A6, GSR, S100A8, HSPA5, YWHAE and CNDP2 and/or decreased level of one or more biomarkers selected from the group consisting of CST1, CST4, CST2, B2M, CST5 and CST3 and/or normal level of either one of both of CSTA, CSTB, as compared to a relevant control. These further biomarkers may be used in any desired combination.

Accordingly, in addition to basing the prediction on PROL1 as set forth above, a subject who is likely to benefit from the treatment, such as a drug switch (e.g. from a preservative-containing eye drug to preservative-free eye drug), to some extent (i.e. patient group 2) may be identified by increased level of one or more biomarkers selected from the group consisting of UCHL3, RNH1, CSTB, CST5 and CST2 and/or decreased level of MYL6 and/or normal level one or more biomarkers selected from the group consisting of TF, CST4, HSPA8, CST1, YWHAZ, S100A6, B2M, GSR, S100A8, HSPA5, YWHAE, CNDP2, CSTA and CST3 as compared to a relevant control. These further biomarkers may be used in any desired combination.

Accordingly, in addition to basing the prediction on PROL1 as set forth above, a subject who is likely not to benefit from the treatment or even experience adverse effects caused by the treatment, such as a drug switch (e.g. from a preservative-containing eye drug to preservative-free eye drug) (i.e. patient group 3) may be identified by increased level of one or more biomarkers selected from the group consisting of CST1, CSTB, CSTA, CST3, CST5, CST2 and CST4 and/or decreased level of MYL6 and/or normal level one or more biomarkers selected from the group consisting of TF, HSPA8, YWHAZ, RNH1, UCHL3, S100A6, B2M, GSR, S100A8, HSPA5, YWHAE and CNDP2 as compared to a relevant control. These further biomarkers may be used in any desired combination.

In accordance with the above, non-limiting examples of preferred biomarker combinations are listed Table 1 below.

TABLE 1
List of preferred biomarker combinations

| | | | | | | |
|---|---|---|---|---|---|---|
| TF, PROL1 | CST4, | HSPA8, | PROL1, | PROL1, | PROL1, | PROL1, |
| | PROL1 | PROL1 | CST1 | YWHAZ | RNH1 | UCHL3 |
| PROL1, | PROL1, | PROL1, | PROL1, | PROL1, | PROL1, | PROL1, |
| CST2 | MYL6 | S100A6 | B2M | GSR | S100A8 | HSPA5 |
| PROL1, | PROL1, | PROL1, | PROL1, | PROL1, | PROL1, | TF, CST4, |
| YWHAE | CNDP2 | CSTA | CSTB | CST5 | CST3 | PROL1 |
| TF, HSPA8, | TF, PROL1, | TF, PROL1, | TF, PROL1, | TF, PROL1, | TF, PROL1, | TF, PROL1, |
| PROL1 | CST1 | YWHAZ | RNH1 | UCHL3 | CST2 | MYL6 |
| TF, PROL1, | TF, PROL1, | TF, PROL1, | TF, PROL1, | TF, PROL1, | TF, PROL1, | TF, PROL1, |
| S100A6 | B2M | GSR | S100A8 | HSPA5 | YWHAE | CNDP2 |
| TF, PROL1, | TF, PROL1, | TF, PROL1, | TF, PROL1, | CST4, | HSPA8, | PROL1, |
| CSTA | CSTB | CST5 | CST3 | PROL1, | PROL1, | CST1, MYL6 |
| | | | | MYL6 | MYL6 | |
| PROL1, | PROL1, | PROL1, | PROL1, | PROL1, | PROL1, | PROL1, |
| YWHAZ, | RNH1, | UCHL3, | CST2, MYL6 | MYL6, | MYL6, B2M | MYL6, GSR |
| MYL6 | MYL6 | MYL6 | | S100A6 | | |
| PROL1, | PROL1, | PROL1, | PROL1, | PROL1, | PROL1, | PROL1, |
| MYL6, | MYL6, | MYL6, | MYL6, | MYL6, CSTA | MYL6, CSTB | MYL6, CST5 |
| S100A8 | HSPA5 | YWHAE | CNDP2 | | | |
| PROL1, | TF, CST4, | TF, CST4, | TF, CST4, | TF, CST4, | TF, CST4, | TF, CST4, |
| MYL6, CST3 | HSPA8, | PROL1, | PROL1, | PROL1, | PROL1, | PROL1, |
| | PROL1 | CST1 | YWHAZ | RNH1 | UCHL3 | CST2 |
| TF, CST4, | TF, CST4, | TF, CST4, | TF, CST4, | TF, CST4, | TF, CST4, | TF, CST4, |
| PROL1, | PROL1, | PROL1, | PROL1, GSR | PROL1, | PROL1, | PROL1, |
| MYL6 | S100A6 | B2M | | S100A8 | HSPA5 | YWHAE |
| TF, CST4, | TF, CST4, | TF, CST4, | TF, CST4, | TF, CST4, | TF, HSPA8, | TF, HSPA8, |
| PROL1, | PROL1, | PROL1, | PROL1, | PROL1, | PROL1, | PROL1, |
| CNDP2 | CSTA | CSTB | CST5 | CST3 | CST1 | YWHAZ |
| TF, HSPA8, | TF, HSPA8, | TF, HSPA8, | TF, HSPA8, | TF, HSPA8, | TF, HSPA8, | TF, HSPA8, |
| PROL1, | PROL1, | PROL1, | PROL1, | PROL1, | PROL1, | PROL1, GSR |
| RNH1 | UCHL3 | CST2 | MYL6 | S100A6 | B2M | |
| TF, HSPA8, | TF, HSPA8, | TF, HSPA8, | TF, HSPA8, | TF, HSPA8, | TF, HSPA8, | TF, HSPA8, |
| PROL1, | PROL1, | PROL1, | PROL1, | PROL1, | PROL1, | PROL1, |
| S100A8 | HSPA5 | YWHAE | CNDP2 | CSTA | CSTB | CST5 |
| TF, HSPA8, | TF, PROL1, | TF, PROL1, | TF, PROL1, | TF, PROL1, | TF, PROL1, | TF, PROL1, |
| PROL1, | CST1, | CST1, RNH1 | CST1, | CST1, CST2 | CST1, MYL6 | CST1, |
| CST3 | YWHAZ | | UCHL3 | | | S100A6 |
| TF, PROL1, | TF, PROL1, | TF, PROL1, | TF, PROL1, | TF, PROL1, | TF, PROL1, | TF, PROL1, |
| CST1, B2M | CST1, GSR | CST1, | CST1, | CST1, | CST1, | CST1, CSTA |
| | | S100A8 | HSPA5 | YWHAE | CNDP2 | |
| TF, PROL1, | TF, PROL1, | TF, PROL1, | TF, PROL1, | TF, PROL1, | TF, PROL1, | TF, PROL1, |
| CST1, CSTB | CST1, CST5 | CST1, CST3 | YWHAZ, | YWHAZ, | YWHAZ, | YWHAZ, |
| | | | RNH1 | UCHL3 | CST2 | MYL6 |
| TF, PROL1, | TF, PROL1, | TF, PROL1, | TF, PROL1, | TF, PROL1, | TF, PROL1, | TF, PROL1, |
| YWHAZ, | YWHAZ, | YWHAZ, | YWHAZ, | YWHAZ, | YWHAZ, | YWHAZ, |
| S100A6 | B2M | GSR | S100A8 | HSPA5 | YWHAE | CNDP2 |
| TF, PROL1, | TF, PROL1, | TF, PROL1, | TF, PROL1, | TF, PROL1, | TF, PROL1, | TF, PROL1, |
| YWHAZ, | YWHAZ, | YWHAZ, | YWHAZ, | RNH1, | RNH1, CST2 | RNH1, |
| CSTA | CSTB | CST5 | CST3 | UCHL3 | | MYL6 |
| TF, PROL1, | TF, PROL1, | TF, PROL1, | TF, PROL1, | TF, PROL1, | TF, PROL1, | TF, PROL1, |
| RNH1, | RNH1, B2M | RNH1, GSR | RNH1, | RNH1, | RNH1, | RNH1, |
| S100A6 | | | S100A8 | HSPA5 | YWHAE | CNDP2 |
| TF, PROL1, | TF, PROL1, | TF, PROL1, | TF, PROL1, | TF, PROL1, | TF, PROL1, | TF, PROL1, |
| RNH1, CSTA | RNH1, CSTB | RNH1, CST5 | RNH1, CST3 | UCHL3, | UCHL3, | UCHL3, |
| | | | | CST2 | MYL6 | S100A6 |
| TF, PROL1, | TF, PROL1, | TF, PROL1, | TF, PROL1, | TF, PROL1, | TF, PROL1, | TF, PROL1, |
| UCHL3, | UCHL3, GSR | UCHL3, | UCHL3, | UCHL3, | UCHL3, | UCHL3, |
| B2M | | S100A8 | HSPA5 | YWHAE | CNDP2 | CSTA |
| TF, PROL1, | TF, PROL1, | TF, PROL1, | TF, PROL1, | TF, PROL1, | TF, PROL1, | TF, PROL1, |
| UCHL3, | UCHL3, | UCHL3, | CST2, MYL6 | CST2, | CST2, B2M | CST2, GSR |
| CSTB | CST5 | CST3 | | S100A6 | | |
| TF, PROL1, | TF, PROL1, | TF, PROL1, | TF, PROL1, | TF, PROL1, | TF, PROL1, | TF, PROL1, |
| CST2, | CST2, | CST2, | CST2, | CST2, CSTA | CST2, CSTB | CST2, CST5 |
| S100A8 | HSPA5 | YWHAE | CNDP2 | | | |
| TF, PROL1, | TF, PROL1, | TF, PROL1, | TF, PROL1, | TF, PROL1, | TF, PROL1, | TF, PROL1, |
| CST2, CST3 | MYL6, | MYL6, B2M | MYL6, GSR | MYL6, | MYL6, | MYL6, |
| | S100A6 | | | S100A8 | HSPA5 | YWHAE |
| TF, PROL1, | TF, PROL1, | TF, PROL1, | TF, PROL1, | TF, PROL1, | TF, PROL1, | TF, PROL1, |
| MYL6, | MYL6, CSTA | MYL6, CSTB | MYL6, CST5 | MYL6, CST3 | S100A6, | S100A6, |
| CNDP2 | | | | | B2M | GSR |
| TF, PROL1, | TF, PROL1, | TF, PROL1, | TF, PROL1, | TF, PROL1, | TF, PROL1, | TF, PROL1, |
| S100A6, | S100A6, | S100A6, | S100A6, | S100A6, | S100A6, | S100A6, |
| S100A8 | HSPA5 | YWHAE | CNDP2 | CSTA | CSTB | CST5 |
| TF, PROL1, | TF, PROL1, | TF, PROL1, | TF, PROL1, | TF, PROL1, | TF, PROL1, | TF, PROL1, |
| S100A6, | B2M, GSR | B2M, | B2M, | B2M, | B2M, | B2M, CSTA |
| CST3 | | S100A8 | HSPA5 | YWHAE | CNDP2 | |
| TF, PROL1, | TF, PROL1, | TF, PROL1, | TF, PROL1, | TF, PROL1, | TF, PROL1, | TF, PROL1, |
| B2M, CSTB | B2M, CST5 | B2M, CST3 | GSR, | GSR, HSPA5 | GSR, | GSR, CNDP2 |
| | | | S100A8 | | YWHAE | |
| TF, PROL1, | TF, PROL1, | TF, PROL1, | TF, PROL1, | TF, PROL1, | TF, PROL1, | TF, PROL1, |
| GSR, CSTA | GSR, CSTB | GSR, CST5 | GSR, CST3 | S100A8, | S100A8, | S100A8, |
| | | | | HSPA5 | YWHAE | CNDP2 |
| TF, PROL1, | TF, PROL1, | TF, PROL1, | TF, PROL1, | TF, PROL1, | TF, PROL1, | TF, PROL1, |
| S100A8, | S100A8, | S100A8, | S100A8, | HSPA5, | HSPA5, | HSPA5, |
| CSTA | CSTB | CST5 | CST3 | YWHAE | CNDP2 | CSTA |
| TF, PROL1, | TF, PROL1, | TF, PROL1, | TF, PROL1, | TF, PROL1, | TF, PROL1, | TF, PROL1, |
| HSPA5, | HSPA5, | HSPA5, | YWHAE, | YWHAE, | YWHAE, | YWHAE, |
| CSTB | CST5 | CST3 | CNDP2 | CSTA | CSTB | CST5 |
| TF, PROL1, | TF, PROL1, | TF, PROL1, | TF, PROL1, | TF, PROL1, | TF, PROL1, | TF, PROL1, |
| YWHAE, | CNDP2, | CNDP2, | CNDP2, | CNDP2, | CSTA, CSTB | CSTA, CST5 |
| CST3 | CSTA | CSTB | CST5 | CST3 | | |
| TF, PROL1, | TF, PROL1, | TF, PROL1, | TF, PROL1, | CST4, | CST4, | CST4, |
| CSTA, CST3 | CSTB, CST5 | CSTB, CST3 | CST5, CST3 | HSPA8, | PROL1, | PROL1, |
| | | | | PROL1, | CST1, MYL6 | YWHAZ, |
| | | | | MYL6 | | MYL6 |
| CST4, | CST4, | CST4, | CST4, | CST4, | CST4, | CST4, |
| PROL1, | PROL1, | PROL1, | PROL1, | PROL1, | PROL1, | PROL1, |
| RNH1, | UCHL3, | CST2, MYL6 | MYL6, | MYL6, B2M | MYL6, GSR | MYL6, |
| MYL6 | MYL6 | | S100A6 | | | S100A8 |
| CST4, | CST4, | CST4, | CST4, | CST4, | CST4, | CST4, |
| PROL1, | PROL1, | PROL1, | PROL1, | PROL1, | PROL1, | PROL1, |
| MYL6, | MYL6, | MYL6, | MYL6, CSTA | MYL6, CSTB | MYL6, CST5 | MYL6, CST3 |
| HSPA5 | YWHAE | CNDP2 | | | | |
| HSPA8, | HSPA8, | HSPA8, | HSPA8, | HSPA8, | HSPA8, | HSPA8, |
| PROL1, | PROL1, | PROL1, | PROL1, | PROL1, | PROL1, | PROL1, |
| CST1, MYL6 | YWHAZ, | RNH1, | UCHL3, | CST2, MYL6 | MYL6, | MYL6, B2M |
| | MYL6 | MYL6 | MYL6 | | S100A6 | |
| HSPA8, | HSPA8, | HSPA8, | HSPA8, | HSPA8, | HSPA8, | HSPA8, |
| PROL1, | PROL1, | PROL1, | PROL1, | PROL1, | PROL1, | PROL1, |
| MYL6, GSR | MYL6, | MYL6, | MYL6, | MYL6, | MYL6, CSTA | MYL6, CSTB |
| | S100A8 | HSPA5 | YWHAE | CNDP2 | | |
| HSPA8, | HSPA8, | PROL1, | PROL1, | PROL1, | PROL1, | PROL1, |
| PROL1, | PROL1, | CST1, | CST1, | CST1, | CST1, CST2, | CST1, MYL6, |
| MYL6, CST5 | MYL6, CST3 | YWHAZ, | RNH1, | UCHL3, | MYL6 | S100A6 |
| | | MYL6 | MYL6 | MYL6 | | |
| PROL1, | PROL1, | PROL1, | PROL1, | PROL1, | PROL1, | PROL1, |
| CST1, MYL6, | CST1, MYL6, | CST1, MYL6, | CST1, MYL6, | CST1, MYL6, | CST1, MYL6, | CST1, MYL6, |
| B2M | GSR | S100A8 | HSPA5 | YWHAE | CNDP2 | CSTA |
| PROL1, | PROL1, | PROL1, | PROL1, | PROL1, | PROL1, | PROL1, |
| CST1, MYL6, | CST1, MYL6, | CST1, MYL6, | YWHAZ, | YWHAZ, | YWHAZ, | YWHAZ, |
| CSTB | CST5 | CST3 | RNH1, | UCHL3, | CST2, MYL6 | MYL6, |
| | | | MYL6 | MYL6 | | S100A6 |
| PROL1, | PROL1, | PROL1, | PROL1, | PROL1, | PROL1, | PROL1, |
| YWHAZ, | YWHAZ, | YWHAZ, | YWHAZ, | YWHAZ, | YWHAZ, | YWHAZ, |
| MYL6, B2M | MYL6, GSR | MYL6, | MYL6, | MYL6, | MYL6, | MYL6, CSTA |
| | | S100A8 | HSPA5 | YWHAE | CNDP2 | |
| PROL1, | PROL1, | PROL1, | PROL1, | PROL1, | PROL1, | PROL1, |
| YWHAZ, | YWHAZ, | YWHAZ, | RNH1, | RNH1, | RNH1, | RNH1, |
| MYL6, CSTB | MYL6, CST5 | MYL6, CST3 | UCHL3, | CST2, MYL6 | MYL6, | MYL6, B2M |
| | | | MYL6 | | S100A6 | |
| PROL1, | PROL1, | PROL1, | PROL1, | PROL1, | PROL1, | PROL1, |
| RNH1, | RNH1, | RNH1, | RNH1, | RNH1, | RNH1, | RNH1, |
| MYL6, GSR | MYL6, | MYL6, | MYL6, | MYL6, | MYL6, CSTA | MYL6, CSTB |
| | S100A8 | HSPA5 | YWHAE | CNDP2 | | |
| PROL1, | PROL1, | PROL1, | PROL1, | PROL1, | PROL1, | PROL1, |
| RNH1, | RNH1, | UCHL3, | UCHL3, | UCHL3, | UCHL3, | UCHL3, |
| MYL6, CST5 | MYL6, CST3 | CST2, MYL6 | MYL6, | MYL6, B2M | MYL6, GSR | MYL6, |
| | | | S100A6 | | | S100A8 |
| PROL1, | PROL1, | PROL1, | PROL1, | PROL1, | PROL1, | PROL1, |
| UCHL3, | UCHL3, | UCHL3, | UCHL3, | UCHL3, | UCHL3, | UCHL3, |
| MYL6, | MYL6, | MYL6, | MYL6, CSTA | MYL6, CSTB | MYL6, CST5 | MYL6, CST3 |
| HSPA5 | YWHAE | CNDP2 | | | | |
| PROL1, | PROL1, | PROL1, | PROL1, | PROL1, | PROL1, | PROL1, |
| CST2, MYL6, | CST2, MYL6, | CST2, MYL6, | CST2, MYL6, | CST2, MYL6, | CST2, MYL6, | CST2, MYL6, |
| S100A6 | B2M | GSR | S100A8 | HSPA5 | YWHAE | CNDP2 |
| PROL1, | PROL1, | PROL1, | PROL1, | PROL1, | PROL1, | PROL1, |
| CST2, MYL6, | CST2, MYL6, | CST2, MYL6, | CST2, MYL6, | MYL6, | MYL6, | MYL6, |
| CSTA | CSTB | CST5 | CST3 | S100A6, | S100A6, | S100A6, |
| | | | | B2M | GSR | S100A8 |
| PROL1, | PROL1, | PROL1, | PROL1, | PROL1, | PROL1, | PROL1, |
| MYL6, | MYL6, | MYL6, | MYL6, | MYL6, | MYL6, | MYL6, |
| S100A6, | S100A6, | S100A6, | S100A6, | S100A6, | S100A6, | S100A6, |
| HSPA5 | YWHAE | CNDP2 | CSTA | CSTB | CST5 | CST3 |
| PROL1, | PROL1, | PROL1, | PROL1, | PROL1, | PROL1, | PROL1, |
| MYL6, B2M, | MYL6, B2M, | MYL6, B2M, | MYL6, B2M, | MYL6, B2M, | MYL6, B2M, | MYL6, B2M, |
| GSR | S100A8 | HSPA5 | YWHAE | CNDP2 | CSTA | CSTB |
| PROL1, | PROL1, | PROL1, | PROL1, | PROL1, | PROL1, | PROL1, |
| MYL6, B2M, | MYL6, B2M, | MYL6, GSR, | MYL6, GSR, | MYL6, GSR, | MYL6, GSR, | MYL6, GSR, |
| CST5 | CST3 | S100A8 | HSPA5 | YWHAE | CNDP2 | CSTA |
| PROL1, | PROL1, | PROL1, | PROL1, | PROL1, | PROL1, | PROL1, |
| MYL6, GSR, | MYL6, GSR, | MYL6, GSR, | MYL6, | MYL6, | MYL6, | MYL6, |
| CSTB | CST5 | CST3 | S100A8, | S100A8, | S100A8, | S100A8, |
| | | | HSPA5 | YWHAE | CNDP2 | CSTA |
| PROL1, | PROL1, | PROL1, | PROL1, | PROL1, | PROL1, | PROL1, |
| MYL6, | MYL6, | MYL6, | MYL6, | MYL6, | MYL6, | MYL6, |
| S100A8, | S100A8, | S100A8, | HSPA5, | HSPA5, | HSPA5, | HSPA5, |
| CSTB | CST5 | CST3 | YWHAE | CNDP2 | CSTA | CSTB |
| PROL1, | PROL1, | PROL1, | PROL1, | PROL1, | PROL1, | PROL1, |
| MYL6, | MYL6, | MYL6, | MYL6, | MYL6, | MYL6, | MYL6, |
| HSPA5, | HSPA5, | YWHAE, | YWHAE, | YWHAE, | YWHAE, | YWHAE, |
| CST5 | CST3 | CNDP2 | CSTA | CSTB | CST5 | CST3 |
| PROL1, | PROL1, | PROL1, | PROL1, | PROL1, | PROL1, | PROL1, |
| MYL6, | MYL6, | MYL6, | MYL6, | MYL6, | MYL6, | MYL6, |
| CNDP2, | CNDP2, | CNDP2, | CNDP2, | CSTA, CSTB | CSTA, CST5 | CSTA, CST3 |
| CSTA | CSTB | CST5 | CST3 | | | |
| PROL1, | PROL1, | PROL1, | | | | |
| MYL6, | MYL6, | MYL6, CST5, | | | | |
| CSTB, CST5 | CSTB, CST3 | CST3 | | | | |

In some embodiments, the present method may comprise a drug switch, i.e. changing of an eye medicament previously used to a different eye medicament. Preferably, a preservative-containing eye medicament is switched to preservative-free eye medicament. In some embodiments, the present method may comprising administering an eye medicament, preferably a preservative-free eye medicament to the subject whose response to an eye disease treatment is to be predicted.

The present biomarker profiles in may be determined by any suitable technique available in the art including, but not limited to any mass spectrometry method (f.ex. microLC-MS/MS), any antibody-based technique (f.ex. ELISA, Western blotting), any RNA-based technique (f.ex. RNA-seq or microarrays).

In one aspect, the present invention relates to a kit for implementing the present methods. Said kit may comprise any reagents or test agents necessary for assessing the level of biomarker combinations disclosed herein. A person skilled in the art can easily determine the reagents to be included depending on the biomarker combination in question and a desired technique for carrying out said assessment. In some embodiments, an appropriate control sample or a threshold value may be comprised in the kit. The kit may also comprise a computer readable medium, comprising computer-executable instructions for performing any of the methods of the present disclosure.

As is apparent to a skilled person any embodiments, details, advantages, etc. of the present disclosure regarding the method of predicting a subject's response to an eye disease treatment apply accordingly to other aspects of the present disclosure, including use of the disclosed biomarker combinations in said prediction and a kit for use in said prediction, and vice versa.

It will be obvious to a person skilled in the art that, as technology advances, the inventive concept can be implemented in various ways. The invention and its embodiments are not limited to the examples described below but may vary within the scope of the claims.

### EXAMPLES

### Example 1. Glaucoma Medication Switch

### Methods

### Study Population

The study was conducted in accordance with the International Conference of Harmonization Good Clinical Practice guidelines and the Declaration of Helsinki. Clinical study was approved by the Ethics Committee at Tampere University Hospital and was registered in EU clinical trials register (EudraCT Number: 2010-021039-14). Each patient signed a written informed consent before inclusion in the study.

The patients were assessed during the baseline visit and eligible patients had primary open angle, capsular glaucoma or ocular hypertension in one or both eyes. The included patients had also been receiving preserved latanoprost treatment for 6 months or longer and exhibited at least two ocular symptoms or one symptom and one sign of ocular surface irritation/inflammation. Thirty patients were selected based on these inclusion criteria.

Patients who were excluded from the study had pigmentary or angleclosure glaucoma, IOP higher than 22 mmHg in baseline, corneal abnormalities affecting tonometry, had undergone a recent (within 6 months) ocular surgery including laser procedures, wore contact lenses, or were using artificial tears containing preservatives. In addition, pregnant and nursing women as well as women of childbearing potential without adequate contraception were excluded.

### Study Outline

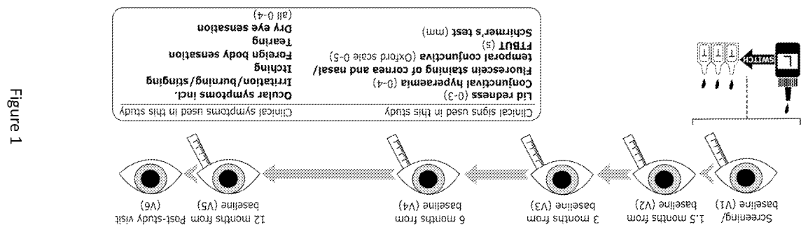

The study consisted of 6 visits: screening/baseline visit, visits at 1.5, 3, 6 and 12 months after the baseline, and 1-4 weeks after the 12-month visit (FIG. 1). In baseline visit, the patients were switched from preserved latanoprost (Xalatan®, Pfizer Inc., New York, N.Y., USA) to preservative-free tafluprost (Taflotan®, Santen, Osaka, Japan) and their backgrounds and medical history were recorded. The new eye drops were administered once a day for the duration of the study. The tear samples for proteomics analysis were collected using a Schirmer's strip and in addition, several ocular examinations and procedures were performed during each visit including ocular symptoms, lid redness, conjunctival hyperaemia, fluorescein staining of cornea and nasal and temporal conjunctiva, fluorescein tear break-up time (FTBUT) and Schirmer's test. The ocular symptoms were graded between 0 and 4 in the following scaling: none (0), trace (1), mild (2), moderate (3) and severe (4). For the ocular signs, conjunctival hyperaemia was assessed using reference photographs and a similar scale as with ocular symptoms. The fluorescein tear break-up time (FTBUT) was evaluated under a slit lamp microscope (seconds) and fluorescein staining of the cornea and nasal and temporal conjunctiva was measured according to the Oxford grading scale from 0 to 5. Lid rednesswas evaluated as none (0), mild (1), moderate (2) and severe (3) and tear secretion was measured using Schirmer's test (mm), which was then used for proteomics analysis. Clinical examinations and sample collections were performed at the same time of the day during each visit. No tear samples were collected during the last visit (V6, ˜12.5 months after baseline).

### Tear Fluid Collection and Sample Preparation

Patient tear fluid samples were collected with Schirmer's strips without anaesthesia (Tear Touch, Madhu Instruments, New Delhi, India). The strips were inserted under patients' lower eye lids and removed after 5 min. Tear amounts (in mm) were recorded and strips were then stored at −80° C. until proteomic analyses.

For extraction of tear proteins, Schirmer's strips were first cut into small pieces and solubilized in 50 mM ammonium bicarbonate solution containing protease inhibitor cocktail (Thermo Fisher Scientific Inc., Waltham, Mass., USA) for 3 h. Samples were then centrifuged and total protein concentration of the supernatants was measured. Up to 50 μg of protein from each sample was dried in a speed vacuum concentrator. Proteins were solubilized in 2% sodium dodecylsulfate (SDS) and reduced by 50 mMTris-(2-carboxyethyl) phosphine (TCEP) for 60 min at +60° C. Samples were then transferred into 30 kDa molecular weight cut-off filters (Pall Corporation, Port Washington, N.Y., USA) and flushed two times with 8 M urea in 50 mM Tris-HCl (Merck KgaA, Darmstadt, Germany). Cysteine residues were blocked by iodoacetamide (IAA) at room temperature in the dark. Alkylation was terminated by centrifugation and the samples were flushed three times with urea solution. Three subsequent rinses with digestion buffer were performed prior to digestion with trypsin (Sciex, Framingham, Mass., USA) for 16 h at +37° C. at a trypsin-to-protein ratio of 1:25. Digests were dried in a speed vacuum concentrator and desalted with Pierce C18 tips (Thermo Fisher Scientific) according to manufacturer's instructions. After clean up the samples were once more vacuum dried and stored at −20° C. until reconstituted to loading solution (5% ACN, 0.1% FA) at equal concentrations. HRM peptide mix (Biognosys, Zurich, Switzerland) was added to each sample before the NanoRPLCtripleTOFT™ SWATH analysis. All reagents were purchased from Sigma-Aldrich (St. Louis, Mo., USA) unless otherwise stated. Two replicate MS analyses were performed from each sample.

Protein Identification and Quantification with SWATH-MS Analysis

Digested peptides were analysed using Eksigent 425 NanoLC coupled with high speed TripleTOFT™ 5600+ mass spectrometer (Ab Sciex, Concord, Canada). A capillary RP-LC column (cHiPLC® ChromXP C18-CL, 3 μm particle size, 120 Å, 75 μm i.d×15 cm, Eksigent Concord, Canada) was used for liquid chromatography separation of peptides. Samples were first loaded into trap column (cHiPLC® ChromXP C18-CL, 3 μm particle size, 120 Å, 75 μm i.d×5 mm) from autosampler and flushed for 10 min at 2 μl/min (2% ACN, 0.1% FA). The flush system was then switched to line with analytical column. Tear samples were analysed with 120 min 6 step gradient using eluent A: 0.1% FA in 1% ACN and eluent B: 0.1% FA in ACN (eluent B from 5% to 7% over 2 min, 7% to 24% over 55 min, 24% to 40% over 29 min, 40% to 60% over 6 min, 60% to 90% over 2 min and kept at 90% for 15 min, 90% to 5% over 0.1 min and kept at 5% for 13 min) at 300 nl/min.

Key parameters for the mass spectrometer in SWATH ID library analysis were: ion spray voltage floating (ISVF) 2300 V, curtain gas (CUR) 30, interface heater temperature (IHT)+125° C., ion source gas 1 13, declustering potential (DP) 100 V. Library for SWATH analysis was created from the same samples by information dependent-acquisition (IDA) method and relative quantitation analysis was done by SWATH method. All methods were controlled by Analyst TF 1.5 software (Ab Sciex, USA). For IDA parameters, 0.25 s MS survey scan in the mass range 350-1250 mz followed by 60 MS/MS scans in the mass range of 100-1500 Da (total cycle time 3.302 s). Switching criteria were set to ions with mass to charge ratio (m/z) greater than 350 and smaller than 1250 (m/z), with charge state 2-5 and an abundance threshold of more than 120 counts. Exclusion of former target ions was set for 12 s. IDA rolling collision energy (CE) parameters script was set for automatically controlling CE. SWATH quantification analysis parameters were the same as for SWATH ID, with the following exceptions: cycle time 3.332 s and MS parameters set to 15 Da windows with 1 Da overlap between mass range 350-1250 Da followed by 40 MS/MS scans in the mass range of 100-1500 Da.

### SWATH Library Creation

SWATH library was created with Protein pilot software version 4.6 (Sciex, Canada) which was used to analyze MS/MS data and searched against the Uniprot Swissprot confidential library for protein identification. Some important settings in the Paragon search algorithm in protein pilot were configured as follows. Sample type: identification, Cys-alkylation: IAA, Digestion: Trypsin, Instrument: TripleTOF 5600+, Search effort: thorough ID. False discovery rate (FDR) analysis was performed in the Protein pilot and FDR<1% was set for protein identification. The data from all the identification runs from patients were combined as a batch and used for library creation. PeakView® software 2.0 with SWATH was used to assign the correct peaks to correct peptides in the library. iRT peptides (Biognosys, Switzerland) was used for retention time calibration with PeakView. 1-12 peptides per protein and 5 transitions per peptide were selected to be used in SWATH quantification. All shared peptides were excluded from analysis. SWATH plug-in FDR analysis was used to select the proper peptides for use in quantification. All proteins with significant or interesting findings in the data analysis were subjected to manual inspection of peptides. This consisted of checking correct peak selection in the chromatogram (FDR 1%, 99% peptide confidence level), sufficient signal to noise ratio inspection (>7) and chromatogram inspection in relation to library chromatogram. Also variation of replicate injections were calculated by means to all samples/protein. All peptides were eliminated from results processing if manual inspection requirements were not fulfilled. Relevant information on SWATH library are in the supplementary data.

### Data Processing and Statistical Analysis

Altogether, 785 proteins were successfully quantified using the SWATH-MS method. Log 2-transformation was applied to achieve more normally distributed data and quantile normalization was implemented. The majority of the samples had two replicate MS analyses and the variation between them was evaluated by intraclass correlation (ICC package in R) and by permutation tests using Spearman's rank correlation coefficients. The replicate MS analyses were then combined by taking geometric means.

For the clinical data, paired t-test for continuous and paired 2-group Wilcoxon Signed Rank Test for ordinal clinical signs and symptoms were used to test how the clinical signs and symptoms changed during visits. For proteomic data fold changes (log 2) between baseline and other visits were analysed using hierarchical clustering (Euclidean distance measure and Ward's method as the criterion) in order to identify clustered groups of proteins with association to ocular surface complications. The clusters of interest were identified using Ingenuity® Pathway Analysis (IPA) and confirmed by identifying well-known biomarkers. The chosen protein clusters were used to separate patients to groups based on their improved or worsened protein expression levels, again using hierarchical clustering method. ANOVA was used to establish proteins, which could separate these patient groups based on their baseline expression levels alone. Pairwise comparisons were conducted to the statistically significant results which did not suffer from heteroscedasticity according to Levene's test for homogeneity of variance (p-value>0.05). The linear relationship between identified proteins and the clinical signs was measured either my mixed model regression (lmer function from lme4 package in R) or cumulative link mixed model (clmm function from ordinal package in R) in order to account for the repeated measures from the same patients.

Manual peak checking was implemented on the proteins of interest and as a result, some statistically significant proteins are omitted from the results due to poor peptide matching. Benjamini & Hochberg correction was applied to pvalues and only proteins with an adjusted p-value below threshold (alpha=0.05) were considered unless otherwise stated. All statistical analyses for the proteomics data were performed using R software version 3.2.3 (R Core Team. Foundation for Statistical Computing, Vienna, Austria) and QIAGEN's Ingenuity® Pathway Analysis (IPA®, QIAGEN Redwood City, USA).

### Results

### Study Population Characteristics and Clinical Results

The study population consisted of 28 patients (7 men and 21 women) since one patient died during the follow-up and one discontinued the study. Twenty-five patients were diagnosed with primary open-angle glaucoma and 3 with capsular glaucoma. The mean age of the patients in the beginning of the study was 67.4 years (95% CI: 64.5-70.3). The patients had been on preserved latanoprost treatment for 7.7 years on average (95% CI: 6.1-9.2). We did not find connections between clinical and proteomic results and the patients' age, gender, diagnosis and duration of latanoprost treatment (data not shown).

The majority of the clinical signs and symptoms of the patients continued to improve throughout the 12 months after the switch (Table 2 and Table 3). More specifically, the conjunctival redness and blepharitis decreased while FTBUT and Schirmer's test results increased. These suggest that most patients experienced relief in their adverse effects. Although the corneal and conjunctival staining scores did not change significantly, the overall means suggest that the scores decreased. The most notable decrease in corneal and conjunctiva staining occurred among the patients with the highest initial staining score (not shown). All the symptoms experienced by the patients improved after the switch, although some of these improvements were not statistically significant.

TABLE 2
Changes in clinical signs between baseline and the visits after
medication switch

| | | Baseline | Change from baseline | | | |
|---|---|---|---|---|---|---|
| Clinical sign | | values | 1.5 months | 3 months | 6 months | 12 months |
| Conjunctival | Mean | 1.750 | −0.286 | −0.607 | −0.75 | −0.964 |
| redness | 95% CI | [1.459, 2.041] | [−0.541, −0.03] | [−0.873, −0.341] | [−1.022, −0.478] | [−1.188, −0.741] |
| | P† | | 0.04* | <0.001*** | <0.001*** | <0.001*** |
| Lid redness | Mean | 0.857 | −0.357 | −0.214 | −0.464 | −0.519 |
| | 95% CI | [0.605, 1.109] | [−0.574, −0.14] | [−0.48, 0.052] | [−0.733, −0.196] | [−0.836, −0.201] |
| | P† | | 0.004** | 0.12 | 0.003** | 0.005** |
| Fluorescein | Mean | 5.393 | 1.786 | 2.179 | 3.214 | 4.444 |
| tear | 95% CI | [4.284, 6.501] | [0.453, 3.118] | [0.735, 3.622] | [1.531, 4.897] | [2.184, 6.705] |
| break−up | P†† | | 0.01* | 0.005** | <0.001*** | <0.001*** |
| time | | | | | | |
| Corneal | Mean | 0.607 | −0.429 | −0.357 | −0.286 | −0.393 |
| staining | 95% CI | [0.252, 0.963] | [−0.785, −0.072] | [−0.741, 0.026] | [−0.732, 0.16] | [−0.792, 0.007] |
| | P† | | 0.02* | 0.08 | 0.25 | 0.06 |
| Conjunctival | Mean | 1.393 | −0.25 | −0.286 | −0.393 | −0.179 |
| staining (nasal) | 95% CI | [1.070, 1.715] | [−0.654, 0.154] | [−0.633, 0.062] | [−0.806, 0.02] | [−0.614, 0.257] |
| | P† | | 0.31 | 0.12 | 0.07 | 0.53 |
| Conjunctival | Mean | 1.571 | −0.321 | −0.393 | −0.464 | −0.357 |
| staining | 95% CI | [1.265, 1.878] | [−0.656, 0.013] | [−0.819, 0.034] | [−0.852, −0.077] | [−0.755, 0.041] |
| (temporal) | P† | | 0.07 | 0.07 | 0.02* | 0.08 |
| Schirmer's test | Mean | 12.000 | 4.214 | 4.964 | 4.786 | 5.429 |
| | 95% CI | [8.511, 15.489] | [1.987, 6.441] | [1.349, 8.579] | [1.288, 8.283] | [2.622, 8.236] |
| | P†† | | <0.001*** | 0.009** | 0.009** | <0.001*** |

*P < 0.05
**P < 0.01
***P < 0.001
†2-group Wilcoxon Signed Rank Test
††Paired t-test
CI (confidence interval)

TABLE 3
Changes in clinical symptoms between baseline and the visits after
medication switch

| Clinical | | | Change from baseline | | | |
|---|---|---|---|---|---|---|
| symptom | | Baseline values | 1.5 months | 3 months | 6 months | 12 months |
| Irritation, | Mean | 1.143 | −0.071 | −0.179 | −0.143 | −0.143 |
| burning, | 95% CI | [0.629, 1.657] | [−0.738, 0.595] | [−0.855, 0.498] | [−0.849, 0.563] | [−0.737, 0.451] |
| stinging | P† | | 0.73 | 0.65 | 0.61 | 0.66 |
| Itching | Mean | 1.821 | −0.643 | −0.821 | −0.643 | −0.75 |
| | 95% CI | [1.361, 2.282] | [−1.14, −0.145] | [−1.339, −0.304] | [−1.162, −0.124] | [−1.284, −0.216] |
| | P† | | 0.02* | 0.007** | 0.02* | 0.01* |
| Foreign body | Mean | 1.571 | −0.786 | −1 | −0.964 | −0.393 |
| sensation | 95% CI | [1.061, 2.082] | [−1.328, −0.244] | [−1.615, −0.385] | [−1.497, −0.432] | [4.055, 0.269] |
| | P† | | 0.008** | 0.009** | 0.003** | 0.30 |
| Tearing | Mean | 1.893 | −0.929 | −1.036 | −1.393 | −1 |
| | 95% CI | [1.321, 2.464] | [−1.466, −0.391] | [−1.673, −0.398] | [−1.934, −0.851] | [−1.517, −0.483] |
| | P† | | 0.004** | 0.009** | <0.001*** | 0.002** |
| Dry eye | Mean | 2.071 | −0.357 | −0.536 | −0.679 | −0.607 |
| sensation | 95% CI | [1.555, 2.588] | [−0.855, 0.14] | [−0.951, −0.12] | [−1.247, −0.11] | [−1.294, 0.08] |
| | P† | | 0.16 | 0.03* | 0.03* | 0.09 |

*P < 0.05
**P < 0.01
***P < 0.001
2-group Wilcoxon Signed Rank Test
CI (confidence interval)

### Varying Relative Protein Expression Levels can be Used to Stratify Patients

Altogether 785 proteins were quantified from each tear sample with SWATH-MS. The proteomic data exhibited good quality and reliability with p-value<0.05 in 89% of replicate MS analyses (permutation tests, Spearman's rank correlation) and mean ICC coefficient of 0.97.

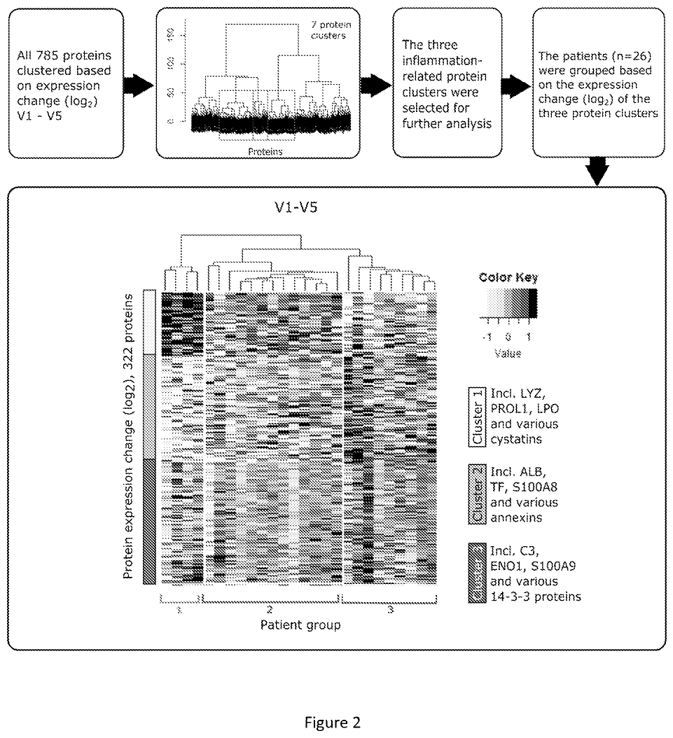

We wanted to establish how each patient's protein profile changes during the 12 month treatment period. To do so, we first clustered the log 2 fold changes between the first and final visit, and based on the dendrogram and visual inspection of results we set the cut-off at 7 clusters (FIG. 2). We then conducted pathway analysis with IPA for all 7 protein clusters and identified three clusters of interest, enriched with inflammatory proteins. Changes in protein expression levels within these three clusters are represented in FIG. 2 including the protein expression data also from earlier visits and displaying well how the protein profiles among patients are clarified during the 12 month follow-up time. The first protein cluster consisted of various beneficial (lacrimal gland secreted proteins) ocular surface biomarkers, such as lysozyme (LYZ), proline-rich protein 1 (PROL1) and various cystatins. Altogether there were 71 proteins in this cluster and the top enriched disease and function terms included activation of neutrophils and chronic inflammatory disorder. The second cluster included inflammation biomarkers such as albumin (ALB), serotransferrin (TF), protein S100A8 and annexins, with a total of 116 proteins all displaying similar fold changes. The top enriched terms for this cluster included inflammation of organ and cell death. The third cluster also included known inflammation biomarkers such as complement C3 (C3), alpha-enolase (ENO1) and protein S100A9. The 135 similarly expressed proteins in this cluster had enrichments relating to cell death, cell movement and cellular infiltration.

Based on the changes in proteomic profiles during the study, it was possible to stratify patients into three groups (FIG. 2). The patients in groups 1 and 2 showed an improvement based on their proteomic profile: antiinflammatory protein expression increased and pro-inflammatory protein expression decreased. The patients in group 3 experienced a decrease in antiinflammatory protein expression and an increase in pro-inflammatory protein expression, suggesting that they were not benefiting as much from the drug switch.

### Baseline Expression Levels of Several Proteins Indicate Individual Differences Between the Patient Groups

Next, we wanted to test if baseline expression levels of specific proteins could explain the differences between the three patient groups at V5 and performed one-way ANOVA. After p-value adjustment, out of 322 clustered proteins, 31 remained statistically significant (p-value<0.05). We excluded one protein without a gene symbol (immunoglobulin), two proteins with unequal variance (heteroscedasticity, Levene's test p-value<0.05) and six proteins with poor peak matches, yielding a total of 22 proteins which differ between the patient groups at the baseline (FIG. 3).

Protein Expression Levels Correlate with Schirmer's Test and FTBUT Values

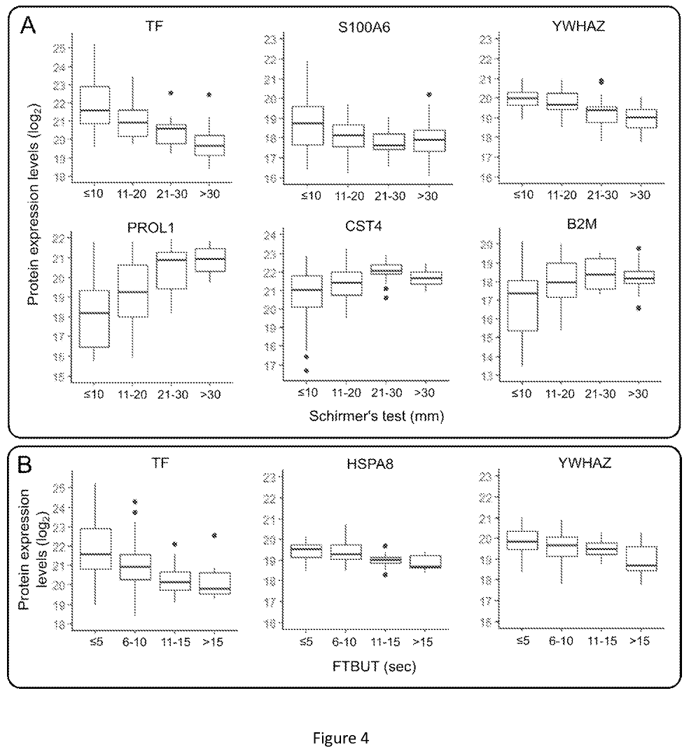

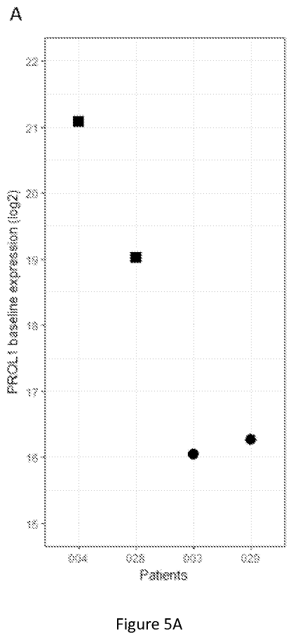

Finally, we wanted to examine clinical results with tear proteomics data in three identified patient groups and performed mixed effects model analysis. All pro-inflammatory proteins that differed between three patient groups (FIG. 3A) correlated with Schirmer's test results, and four correlated with FTBUT (Table 4 and FIG. 4). Various cystatins, PROL1 and B2M (FIG. 3B) correlated positively and acyl-CoA-binding protein (DBI) correlated negatively (FIG. 3C) with Schirmer's test (FIG. 4). We observed that low Schirmer's test and FTBUT values correlate with high expression of harmful pro-inflammatory proteins and low expression of beneficial proteins.

TABLE 4
Proteins differentiating the patient groups at baseline, which also
correlate with Schirmer's test and fluorescein tear break-up time (FTBUT)
The proteins with negative log2(exprs) decrease as the clinical sign is increased
and vice versa for the positive values.

| | | | Coef ficient | P-va | es | |
|---|---|---|---|---|---|---|
| Uniprot | Firl, name | Symbol | intercept | tiN7.expr) | unatki, | adj. |
| P62258 | 14-3-3 protein epsiion | YWHAE | 136.584 | −6.761 | <0.001 | <0.001 |
| P13489 | Ribonuclease inhibitor | RNH1 | 119.600 | −6.454 | <0.001 | <0.001 |
| P11142 | Heat shock cognate 71 kDa | HSPA8 | 132.799 | −6.042 | <0.001 | <0.001 |
| | protein | | | | | |
| P63104 | 14-3-3 protein 20-zeta/delta | YWHAZ | 131.371 | −5.865 | <0.001 | <0.001 |
| P00390 | Glutathione reductase, | GSR | 105.298 | −4.909 | <0.001 | <0.001 |
| | mitochondrial | | | | | |
| P11021 | 78 kDa glucose-regulated protein | HSPA5 | 97.092 | −4.802 | <0.001 | <0.001 |
| P60660 | Myosin light polypeptide 6 | MYL6 | 95.605 | −4.702 | <0.001 | <0.001 |
| P02787 | Serotransferrin | TF | 88.786 | −3.439 | <0.001 | <0.001 |
| P06703 | Protein S100-A6 | S100A6 | 68.254 | −2.849 | <0.001 | <0.001 |
| P07108 | Acyl-CoA-binding | DBI | 53.620 | −2.228 | 0.007 | 0.010 |
| | Ubiiquitin carboxyl-terminal | | | | | |
| P15374 | hydrolase isozyme L3 | UCHL3 | 38.406 | −1.713 | 0.002 | 0.003 |
| P01036 | Cystatin-S | CST4 | −24.043 | 1.772 | 0.002 | 0.003 |
| P09228 | Cystatin-SA | CST2 | −34.424 | 2.365 | <0.001 | <0.001 |
| P61769 | Beta-2-microglobulin | B2M | −25.904 | 2.396 | <0.001 | <0.001 |
| P01037 | Cystatin-SN | CST1 | −39.967 | 2.645 | <0.001 | <0.001 |
| Q99935 | Proline-rich protein 1 | PROL1 | −35.713 | 2.683 | <0.001 | <0.001 |
| P11142 | Heat shock cognate 71 kDa | HSPA8 | 69.072 | −3.181 | <0.001 | 0.010 |
| | protein | | | | | |
| P63104 | 14-3-3 protein zeta/delta | YWHAZ | 42.423 | −1.769 | 0.002 | 0.018 |
| P00390 | Glutathione reductase, | GSR | 33.930 | −1.445 | 0.001 | 0.016 |
| | mitochondrial | | | | | |
| P02787 | Serotransferrin | TF | 27.844 | −0.954 | 0.003 | 0.019 |

DV = dependent variable

### Example 2. Patient Examples

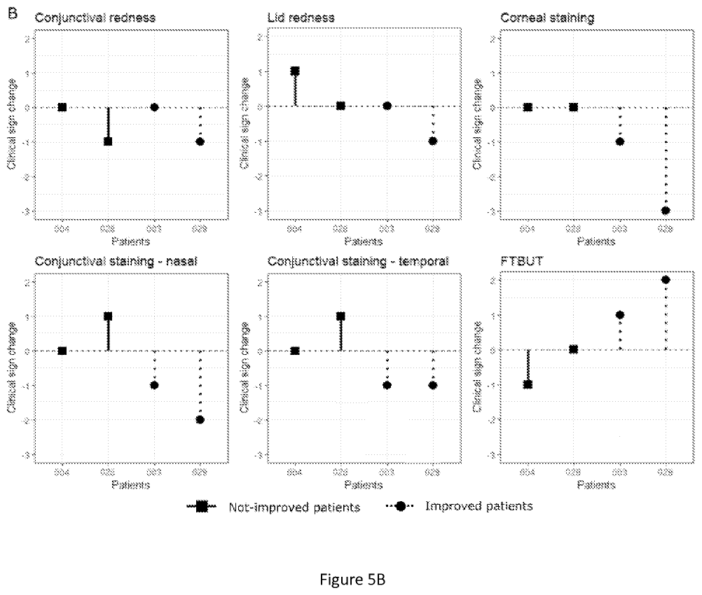

Four patients with similar histories of adverse effects during their preserved prostaglandin analogue eye medication were selected as patient examples herein. Further patient characteristics were also similar: all patients were females with an age range from 62 to 69 years. All four patients were switched from preserved to preservative-free prostaglandin analogue during the study, however, as shown in FIG. 5, two of the patients (003, 029) were benefiting from the switch according to both proteomic profiles and clinical signs after one year, while the other two were not (004, 028). One of the proteins differentiating these patients at baseline was PROL1, which had a lowered expression level among the patients benefiting from the switch according to both clinical and proteomic profile changes.

This example confirms that PROL1 can be used as a predictive biomarker for determining a response to an eye disease medication.

1-13. (canceled)

14. A method of predicting a response to an eye disease treatment in a subject in need thereof, comprising
assessing the level of at least biomarkers comprising PROL1 (Proline-rich protein 1) and MYL6 (Myosin light polypeptide 6) in a sample obtained from said subject,
comparing the assessed level of said biomarkers to a control, and
predicting said subject's response to said treatment on the basis of the comparison, wherein decreased level of PROL1 and increased level of MYL6 is indicative that said subject will benefit from said treatment.

15. The method according to claim 14, wherein the sample is assessed for the level of one or more further biomarkers selected from the group consisting of TF (Serotransferrin), CST4 (Cystatin-S), HSPA8 (Heat shock cognate 71 kDa protein), CST1 (Cystatin-SN), YWHAZ (14-3-3 protein zeta/delta), RNH1 (Ribonuclease inhibitor), UCHL3 (Ubiquitin carboxyl-terminal hydrolase isozyme L3), CST2 (Cystatin-SA), S100A6 (Protein S100-A6), B2M (Beta-2-microglobulin), GSR (Glutathione reductase, mitochondrial), S100A8 (Protein S100-A8), HSPA5 (78 kDa glucose-regulated protein), YWHAE (14-3-3 protein epsilon), CNDP2 (Cytosolic non-specific dipeptidase), CSTA (Cystatin-A), CSTB (Cystatin-B), CST5 (Cystatin-D), and CST3 (Cystatin-C),
wherein increased level of a biomarker selected from the group consisting of TF, HSPA8, YWHAZ, RNH1, UCHL3, S100A6, GSR, S100A8, HSPA5, YWHAE and CNDP2 and/or decreased level of a biomarker selected from the group consisting of CST1, CST4, CST2, B2M, CST5 and CST3 and/or normal level of either one of both of CSTA, CSTB, as compared to a control is indicative that said subject will benefit from the treatment.

16. The method according to claim 15, wherein said further biomarker is TF, whose increased level is indicative that said subject will benefit from said treatment.

17. The method according to claim 15, wherein said further biomarker is CST1, whose decreased level is indicative that said subject will benefit from said treatment.

18. The method according to claim 14, wherein said eye disease is selected from the group consisting of any forms of glaucoma, ocular hypertension, ocular surface disease, ocular infection, iritis and uveitis.

19. The method according to claim 14, wherein said treatment comprises treating said eye disease with a topical eye drop, ointment, gel or any type of drug delivering implant.

20. The method according to claim 19, wherein said treatment comprises a drug switch.

21. Use of the method according to claim 14 for selecting a treatment regime to a subject having an eye disease, monitoring response to treatment in a subject having an eye disease, stratifying subjects on the basis of the predicted response to treatment, or stratifying subjects for clinical trials.

22. Use of a biomarker combination comprising PROL1 and MYL6 and optionally one further biomarker TF for determining a subject's response to an eye disease treatment.

23. The use according to claim 22, wherein the biomarker combination further comprises one or more biomarkers selected from the group consisting of CST4, HSPA8, CST1, YWHAZ, RNH1, UCHL3, CST2, S100A6, B2M, GSR, S100A8, HSPA5, YWHAE, CNDP2, CSTA, CSTB, CST5, and CST3.

24. A kit for use in the method according to claim 14, comprising reagents for specifically assessing the level of PROL1 and MYL6 and optionally one further biomarker TF.

25. The kit according to claim 24 further comprising reagents for specifically assessing the level of one or more biomarkers selected from the group consisting of CST4, HSPA8, CST1, YWHAZ, RNH1, UCHL3, CST2, S100A6, B2M, GSR, S100A8, HSPA5, YWHAE, CNDP2, CSTA, CSTB, CST5, and CST3 in a biological sample, preferably a tear sample.
